(19) United States
(12) Patent Application Publication
(10) Pub. No.: US 2016/0174863 A1
(43) Pub. Date: Jun. 23, 2016

(54) METHOD OF DATA COMPRESSION PREPROCESSING TAILORED TO DATA OF MEASUREMENTS OF ELECTRO-CORTICOGRAPHIC SIGNALS (ECoG) AND SYSTEM FOR ACQUIRING AND TRANSMITTING ECoG DATA

(71) Applicant: COMMISSARIAT A L'ENERGIE ATOMIQUE ET AUX ENERGIES ALTERNATIVES, PARIS (FR)

(72) Inventors: Michael FOERSTER, GRADIGNAN (FR); David DEHAENE, PALAISEAU (FR)

(21) Appl. No.: 14/971,960

(22) Filed: Dec. 16, 2015

(30) Foreign Application Priority Data
Dec. 22, 2014 (FR) 1463136

(51) Int. Cl. A61B 5/04; A61B 5/00; A61B 5/0478

(52) U.S. Cl. CPC A61B 5/04012; A61B 5/0478; A61B 5/0006

(57) ABSTRACT

A method of compression preprocessing of raw data uᵢ(t_{N}) of measurements of electrocorticographic signals (ECoG) evolving over time with the aid of electrodes disposed in direct contact with a cortex comprises an actual step of compression preprocessing in which each raw signal uᵢ(t_{N}) acquired by the observed electrode i is transformed into a preprocessed signal εᵢ (t_{N}) equal to the difference between a first term and a second term and appropriate for a second entropy encoding step. The first term is equal to the raw signal acquired at the electrode i at the current instant t_{N}, and the second term is a prediction function fi which depends on past raw signals observed in a near past at at least neighbour electrodes σᵢ(j) of the observed electrode or at most at neighbour electrodes σᵢ(j) of the observed electrode i and at the observed electrode.

### CROSS-REFERENCE TO RELATED APPLICATION

This application claims priority to foreign French patent application No. FR 1463136, filed on Dec. 22, 2014, the disclosure of which is incorporated by reference in its entirety.

### FIELD OF THE INVENTION

The present invention relates to a method of data compression preprocessing tailored to raw data uᵢ(t_{N}) of measurements of electrocorticographic signals evolving over time, and to a system for implementing such a method.

The invention also relates to a computer program implementing the method and implemented by the system according to the invention.

### BACKGROUND

In the field of the acquisition of cerebral signals on the surface of the cortex of an animal or human being, dubbed the “patient”, it is known and very widespread to acquire the signals by means of an ElectroCorticoGram (ECoG) measurement system.

Systems for acquiring cerebral biological signals have existed for a long time, but it is only recently that miniaturization of the electrodes and the electronics has made it possible to develop wireless acquisition systems. Conventional wired systems, such as EEG electroencephalographic measurement caps for example, physically attach the sensor or sensors in proximity or in direct contact with the cortex of a patient to measurement electronics, the latter being in its turn linked to a data processing unit, for example a computer.

Wireless systems, on the other hand, which are particularly appropriate for the use of electrodes in direct contact with the surface of a cortex, integrate the electronics at the place of the measurement and transmit the data to the data processing unit through a wireless communication means.

This is why, for such wireless systems, the decrease in the volume of the acquired raw data, that is to say their compression, takes on even more significant interest since it makes it possible to reduce the constraints weighing on the wireless data transmission modules or on the data storage media. By extension, it also reduces the energy needs of systems for acquiring cerebral signals and therefore makes it possible to develop systems whose performance is closer to wired systems since fewer design compromises have to be made.

Generally, data compression is a technical field in which, for multiple applications, such as the compression of text, images, sound or videos, a great deal of research has been performed or is ongoing.

The approaches used to reduce the quantity of data depend greatly on the application. Thus, for text compression, dictionary-based approaches are frequently used, where words are substituted by their location in a dictionary, for image or sound compression, a compression with losses will be carried out, while removing the inaudible details, and for video compression, in addition to removing the details which are invisible to the eye, the similarity between two successive images will be exploited.

There exist however common schemes, widely used, which make it possible to carry out data compression while exploiting the statistical properties of the data to be compressed. Huffman coding and arithmetical coding are the most used of these schemes. These techniques often intervene as a supplement to a compression technique tailored to the application.

In sum, a compression algorithm will often apply a preprocessing tailored to the type of data to be processed, before coding them using generic techniques.

In the case of a compression algorithm applied to ECoG measurements data, the compression method comprises a compression preprocessing tailored to data of this type, subsequently dubbed compression preprocessing or preprocessing, followed by a conventional generic entropy compression independent of the application, subsequently dubbed entropy encoding.

In the field of ECoG data compression, the article by Taehoon Kim et al., entitled “Spatiotemporal compression for efficient storage and transmission of high-resolution electrocorticography data”, and published in Proceedings of Engineering in Medicine and Biology Society (EMBC), 2012 Annual International Conference of the IEEE, pp. 1012-1015, 28 Aug. 2012-1 Sep. 2012, describes the application of preprocessing methods arising from the compression of video images to the compression of ECoG ElectroCorticoGram data. The processings chosen demand a great deal of calculation resources and are not devoid of information losses.

The technical problem is to provide a method of data compression preprocessing of ElectroCorticoGram (ECoG) measurements evolving over time which is devoid of ElectroCorticoGram information losses, which faithfully follows with a small delay the temporal evolution of the ElectroCorticoGram (real-time requirement), and which uses a minimum of calculation resources.

In a corresponding manner, the technical problem is to provide a unit for the compression preprocessing of data of ElectroCorticoGram (ECoG) measurements evolving over time which is devoid of ElectroCorticoGram information losses, which faithfully follows with a small delay the temporal evolution of the ElectroCorticoGram, and which uses a minimum of calculation resources, and an acquisition and transmission system comprising such a compressor.

### SUMMARY OF THE INVENTION

For this purpose, the subject of the invention is a method of compression preprocessing of raw data uᵢ(t_{N}) of measurements of electrocorticographic signals evolving over time and acquired with the aid of a set of electrodes disposed in direct contact with a cortex and each characterized by a different integer identification index i and a position r(i) on the surface of the cortex, the index i varying from 1 to L and L designating the total number of the electrodes, the raw electrocorticographic temporal signal uᵢ(t) acquired by the electrode of index i being sampled at successive instants t_{N}, N being an integer designating the succession rank of a sample of the signal over time, as a sampled raw signal uᵢ(t_{N}), the coding of whose amplitude forms the raw acquired data of the electrocorticographic raw signal at the sampling instant t_{N}, characterized in that it comprises an actual step of compression preprocessing of the raw signal uᵢ(t_{N}) consisting in: for i varying from 1 to L and at each successive sampling instant t_{N} onwards of a rank N₀, transforming the raw signal acquired uᵢ(t_{N}) by the observed electrode i into a preprocessed signal εᵢ(t_{N}) equal to the difference between a first term and a second term,

the first term being equal to the raw signal uᵢ(t_{N}) acquired at the electrode i at the current instant t_{N}, and the second term is a prediction function fi which depends temporally on raw signals u_{σ}_{ᵢ}₍ⱼ₎(t_{N-k}), uᵢ(t_{N-k}) observed in a near past up to the instant t_{N-p}, p designating the most distant past rank with respect to the rank N of the current instant, at at least an integer number m of neighbour electrodes σᵢ(j) of the observed electrode i and at most at the integer number m of neighbour electrodes σᵢ(j) of the observed electrode and at the observed electrode i.

According to particular embodiments, the compression preprocessing method comprises one or more of the following characteristics:

the function fi of the second term is a first prediction function f1ᵢ which depends exclusively on the observed raw signals u_{σ}_{ᵢ}₍ⱼ₎(t_{N-k}) in the near past up to the instant t_{N-p}, at the integer number m of neighbour electrodes σᵢ(j) of the observed electrode i, σᵢ being a neighbourhood function of the observed electrode i;

the function fi of the second term is a second prediction function f2ᵢ which depends exclusively on the raw signals u_{σ}_{ᵢ}₍ⱼ₎(t_{N-k}), uᵢ(t_{N-k}), observed in the near past up to the instant t_{N-p}, at the integer number m of neighbour electrodes σᵢ(j) of the observed electrode i and at the observed electrode i, σᵢ being a neighbourhood function of the observed electrode i;

the function fi is a linear prediction function f1Lᵢ which depends exclusively on the raw signals observed in the near past up to the instant t_{N-p}, at the integer number m of neighbour electrodes σᵢ(j) of the observed electrode i or a linear function f2Lᵢ which depends exclusively on the raw signals observed in the near past up to the instant t_{N-p}, at the integer number m of neighbour electrodes σᵢ(j) of the observed electrode and at the observed electrode i;

at least one of the integers m, p is independent of the index i of the observed electrode, or both integers m and p are independent of the index i of the observed electrode;

the second term is a prediction function which depends on raw signals observed in a near past up to the instant t_{N-p}, p designating the most distant past rank with respect to the rank N of the current instant, at an integer number m of immediate neighbour electrodes σᵢ(j) of the observed electrode i;

the method comprises a learning step, executed either just once and for all for the set of observed electrodes i, i varying from 1 to L, before the actual step of compression preprocessing of the raw data, or in a manner spread over time per packet of electrodes and in parallel with the actual step of compression preprocessing of the raw data into preprocessed data, and in the course of which a set of parameters characterizing the prediction functions fi are determined by adjusting them through a statistical processing, the size of the statistic being dependent on a sufficiently large number of temporal samples;

the parameters of the linear prediction functions are matrices of coefficients of linear transformations.

The subject of the invention is also a decompression method corresponding to the compression preprocessing method defined hereinabove, comprising the steps consisting in:

in a first initialization step, receiving for each observed electrode i a suite of initialization data formed by raw signals observed in a near past up to the instant t_{N0-p}, p designating the most distant past rank with respect to a rank N₀ of the start instant of the actual implementation of the compression, at an integer number m of neighbour electrodes σᵢ(j) of the observed electrode i, the suite of data being transmitted through a first link; then

in an iterative running loop of a current rank N, for the current rank N onwards of N₀, and for each electrode i,

in a second step, providing raw signals observed in a near past up to the instant t_{N-p}, p designating the most distant past rank with respect to the rank N of the current instant, at the integer number m of neighbour electrodes σᵢ(j) of the observed electrode or at the integer number m of neighbour electrodes σᵢ(j) of the observed electrode i and the observed electrode i, transmitted directly through the first link or reconstructed on the basis of corresponding relevant compressed signals transmitted through a second link; then

in a third step, receiving the preprocessed datum εᵢ(t_{N}) of the signal acquired at the sampling instant t_{N}, at the electrode i, the said preprocessed datum having been determined by the implementation of the compression preprocessing method defined hereinabove, and having been transmitted through a second link; then

in a fourth step, determining a second reconstruction term on the basis of the prediction function fi of the second term, applied to the raw signals observed in a near past up to the instant t_{N-p}, at the integer number m of neighbour electrodes σᵢ(j) of the observed electrode i or at the integer number m of neighbour electrodes σᵢ(j) of the observed electrode i and the observed electrode i, the observed raw signals being provided beforehand in the second providing step, and then

in a fifth step, reconstructing the raw datum uᵢ(t_{N}) of the signal acquired at the sampling instant t_{N}, as being equal to the sum of the second reconstruction term and of the preprocessed datum εᵢ(t_{N}) of the signal acquired at the sampling instant t_{N}, at the electrode i.

According to particular embodiments, the decompression method comprises one or more of the following characteristics:

the first and second links are the same link, or

the first link is a wired link and the second link is a wireless link.

The subject of the invention is also a unit for transmitting data of measurements of electrocorticographic ECoG signals evolving over time comprising a unit for the compression preprocessing of data for converting raw data uᵢ(t_{N}) of measurements of electrocorticographic signals tapped off by electrodes at diverse locations on the surface of a cortex into compressed preprocessing data εᵢ (t_{N}), and a transmission emitter for transmitting the data preprocessed and then encoded by an entropy coding over a link, characterized in that the compression preprocessing unit is configured to implement the compression preprocessing method defined hereinabove.

According to particular embodiments, the transmission unit comprises one or more of the following characteristics:

the compression preprocessing unit and the transmission emitter are miniaturized so as to be implanted in the body of a patient and the transmission emitter is configured to emit radioelectric waves.

The subject of the invention is also a system for acquiring and transmitting data of measurements of electrocorticographic signals evolving over time comprising:

a set of electrodes disposed in direct contact with a cortex, each characterized by a different integer identification index i and a position r(i) on the surface of the cortex, the index i varying from 1 to L and L designating the total number of electrodes, and each configured to acquire a raw electrocorticographic temporal signal uᵢ(t); and

a sampling unit, configured to sample at successive instants t_{N}, N being an integer designating the succession rank of a sample of a signal over time, the raw signals uᵢ(t) and code in binary their amplitude as associated raw data;

a unit for the compression preprocessing of data for converting the raw data uᵢ(t_{N}) into preprocessed data εᵢ (t_{N}), with lower entropy, and

a transmission emitter for transmitting the compressed preprocessed data; characterized in that the compression preprocessing unit is configured to implement an actual step of compression preprocessing of the raw data uᵢ(t_{N}) consisting in:

for i varying from 1 to L and at each successive sampling instant t_{N} onwards of a rank N₀, transforming the raw signal uᵢ(t_{N}) acquired by the electrode i into a signal of preprocessed εᵢ (t_{N}) equal to the difference between a first term and a second term, the first term being equal to the signal to the raw signal uᵢ(t_{N}) acquired at the electrode i at the current instant t_{N}, and the second term being a prediction function fi which depends temporally on raw signals u_{σ}_{ᵢ}₍ⱼ₎(t_{N-k}), uᵢ(t_{N-k}) observed in a near past up to the instant t_{N-p}, p designating the most distant past rank with respect to the rank N of the current instant, at at least an integer number m of neighbour electrodes σᵢ(j) of the observed electrode i and at most at the integer number m of neighbour electrodes σᵢ(j) of the observed electrode and at the observed electrode i.

According to particular embodiments, the system for acquiring and transmitting data comprises one or more of the following characteristics:

the system also comprises a transmission receiver, remote from the transmission emitter, for receiving the compressed data, and a preprocessing decompressor for decompressing the preprocessed data εᵢ (t_{N}) into the raw data uᵢ(t_{N}), configured to execute the steps of the decompression method which are defined hereinabove.

The subject of the invention is also a computer program or product comprising a set of instructions configured to implement the compression method and/or the decompression method which are defined hereinabove when they are loaded into and executed by one or more processors of the system for acquiring and transmitting data.

### BRIEF DESCRIPTION OF THE DRAWINGS

The invention will be better understood on reading the following description, given solely by way of example, of several embodiments, while referring to the drawings in which:

FIG. 1 is a view of an exemplary architecture of a system for acquiring ECoG measurements and for transmitting ECoG measurements data compressed according to the invention;

FIGS. 2A, 2B, 2C, 2D are illustrations of examples of neighbourhood functions σᵢ, that can be used for the implementation of a method of compression preprocessing of raw ECoG measurements data according to the invention;

FIG. 3 is a flowchart of a method of compression preprocessing of raw ECoG measurements data according to the invention;

FIG. 4 is a view of the entropy-related performance of a compression method using only a conventional entropy encoding and not using any compression preprocessing of raw ECoG measurements data;

FIG. 5 is a view of the entropy-related performance of a compression method using a compression preprocessing according to the invention, applied to raw ECoG measurements data, followed by a generic entropy encoding;

FIG. 6 is a flowchart of a decompression method implemented by the decompressor of the acquisition and transmission system of FIG. 1 and corresponding to a compression preprocessing method according to the invention.

### DETAILED DESCRIPTION

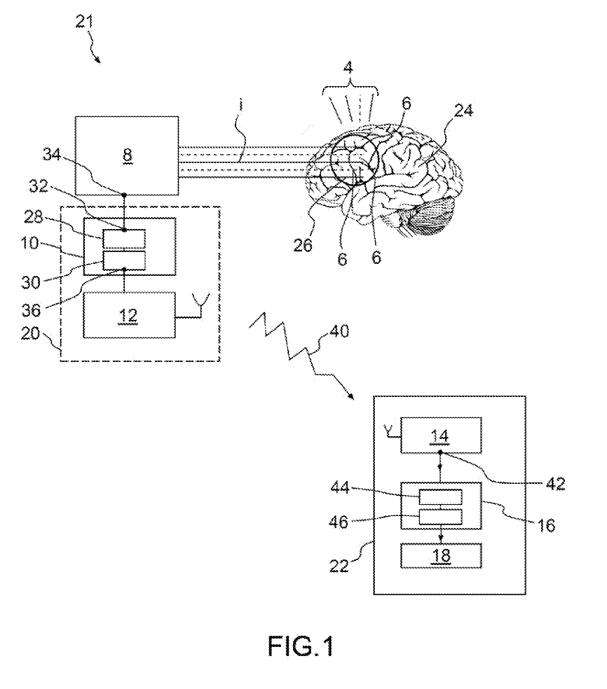

According to FIG. 1, a system 2 for acquiring and transmitting data of measurements of electrocorticographic signals evolving over time comprises a set or a matrix 4 of cortical electrodes 6 for acquiring electrocorticographic raw signals, a unit 8 for sampling the raw signals as raw data, a compressor 10 of the raw data into compressed data to be transmitted, an emitter 12, a receiver 14 and a decompressor of transmitted data 16, and a unit 18 for processing the raw measurements data restored.

The compressor 10 and the emitter 12 are integrated into an emission unit while the receiver 14, the decompressor 16 and the processing unit 18 are integrated into a reception unit 22.

The cortical electrodes 6 of the set or of the matrix 4 are disposed here in direct contact with the cortex 24 of a patient in an observation zone 26.

As a variant, the cortical electrodes are disposed in proximity to the surface of the cortex.

According to FIG. 1, the cortical electrodes 6 are each characterized by a different integer identification index i and a position r(i) on the surface of the cortex, the index i varying from 1 to L, and L designating the total number of electrodes 6.

The cortical electrodes 6 are each configured to acquire a raw electrocorticographic temporal signal designated by uᵢ(t) as a function of the index i of the observed electrode.

The sampling unit 8 is configured to sample at successive instants t_{N}, N being an integer designating the succession rank of a sample of a signal over time, the raw signals uᵢ(t) and code here in binary their amplitude as associated raw data.

Preferably the sampling is performed periodically, and generally the coding of the amplitude is carried out in an arbitrary base.

The data compressor 10 comprises a compression preprocessing unit 28 followed by an entropy encoder 30.

The compression preprocessing unit 28 possesses an input port 32 connected to an output port 34 of the sampling unit 8, and it is configured to convert the raw data uᵢ(t) into preprocessed data εᵢ(t_{N}) with lower entropy according to a compression preprocessing method of the invention.

The transmission emitter 12 is connected to an output port 36 of the compressor 10 of the raw data uᵢ(t), and is configured to transmit the preprocessed data εᵢ(t_{N}) encoded successively by the entropy encoder 30, through a link 22 here wireless, that is to say radioelectric.

The transmission receiver 14, remote from the transmission emitter 12, is configured to receive the preprocessed data εᵢ(t_{N}) encoded successively by the entropy encoder 30, and restore them devoid of error to the decompressor 16, connected to an output port 42 of the transmission receiver 14.

The decompressor 16 comprises an entropy decoder 44 followed by a preprocessing decompressor 46.

The entropy decoder 44, connected to the output port 42 of the transmission receiver 14 and whose generic decompression algorithm corresponds to the generic compression algorithm of the entropy encoder 30, is configured to restore at the input of the preprocessing decompressor 46 the preprocessed data εᵢ(t_{N}) or error signals.

The preprocessing decompressor 46 is configured to decompress the preprocessed data εᵢ(t_{N}) into the raw data uᵢ(t) according to a preprocessing decompression algorithm corresponding to the compression preprocessing algorithm.

The compression preprocessing unit 28 is configured generally to implement a compression preprocessing method which comprises a step of actual compression preprocessing of the raw data.

In this step of actual compression preprocessing of the raw data uᵢ(t), for i varying from 1 to L and at each successive sampling instant t_{N} onwards of a rank N₀, the raw signal uᵢ(t) acquired by the electrode i is transformed into a preprocessed signal or error signal εᵢ(t_{N}), equal to the difference between a first term and a second term.

The first term is equal to the signal to the raw signal acquired at the electrode i at the current instant t_{N}, or of rank N.

The second term is an invertible prediction function fi which depends on raw signals observed in a near past up to the instant t_{N-p}, p designating the most distant past rank with respect to the rank N of the current instant t_{N}, at least at an integer number m of neighbour electrodes σᵢ(j) of the observed electrode i and at most at the integer number m of neighbour electrodes σᵢ(j) of the observed electrode and at the observed electrode i, σᵢ being a neighbourhood function of the observed electrode i which for j varying from 1 to m associates the index σᵢ(j) of a neighbour electrode of the observed electrode i and whose signal is correlated with that of the observed electrode i.

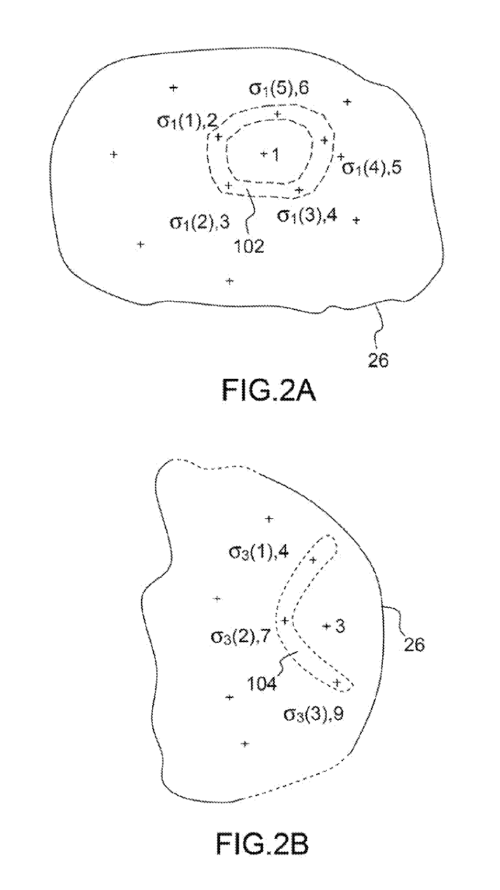

According to FIG. 2A and a first exemplary illustration of a neighbourhood function σᵢ, an observed electrode i is surrounded here by 5 immediate neighbour electrodes according to a single ring 102 illustrated dashed.

Here, the electrode i, observed and represented by way of example in FIG. 2A, is the electrode 1 for which i is equal to 1. The immediate neighbour electrodes are m(1) electrodes in number, m(1) being equal here to 5, the identification indices of these electrodes σ₁(1), σ₁(2), σ₁(3), σ₁(4), σ₁(5) being for this example respectively equal to 2, 3, 4, 5, 6, this correspondence being defined through the neighbourhood function σ₁.

According to FIG. 2B and a second exemplary illustration of a neighbourhood function σᵢ, an observed electrode i, situated at the boundary of the cortical surface zone 26 covered by the set of electrodes, is partially surrounded by immediate neighbour electrodes forming a ring arc 104.

Here, the electrode i observed and represented by way of example in FIG. 2B is the electrode 3 for which i is equal to 3. The immediate neighbour electrodes are m(3) electrodes in number, m(3) being equal here to 3, the identification indices of these electrodes, σ₃(1), σ₃(2), σ₃(3), being respectively equal to 4, 7 and 9, this correspondence being defined through the neighbourhood function σ₃.

According to FIG. 2C and a third exemplary illustration of a neighbourhood function σᵢ, an observed electrode i is surrounded by neighbour electrodes forming two rings 106 and 108.

Here, the electrode i, observed and represented by way of example in FIG. 2C, is the electrode 2 for which i is equal to 2. The neighbour electrodes are m(2) electrodes in number, m(2) being equal here to 8, the identification indices of these electrodes σ₂(1), σ₂(2), σ₂(3), σ₂(4), σ₂(5), σ₂(6), σ₂(7), σ₂(8), being respectively equal to 5, 7, 12, 15, 16, 19, 21, 32, this correspondence being defined through the neighbourhood function σ₂, the electrodes 5, 7, 12 forming a first ring 106, illustrated dashed, of immediate neighbour electrodes surrounding the observed electrode 2, and the electrodes 15, 16, 19, 21, 32, forming a second ring 108, illustrated dashed, of immediate neighbour electrodes surrounding the first ring of electrodes 102.

According to FIG. 2D and a fourth exemplary illustration of a neighbourhood function σᵢ, an observed electrode i, situated at the boundary of the cortical surface zone 24 covered by the set of L electrodes, is partially surrounded by electrodes forming two ring arcs.

Here, the observed electrode i represented by way of example in FIG. 2D is the electrode 5 for which i is equal to 5. The neighbour electrodes are m(5) electrodes in number, m(5) being equal here to 5, the identification indices of these electrodes σ₅(1), σ₅(2), σ₅(3), σ₅(4), σ₅(5), being respectively equal to 7, 8, 17, 20 and 21, this correspondence being defined through the neighbourhood function σ₅, the electrodes 7, 8 forming a first ring arc 112, illustrated dashed, of immediate neighbour electrodes partially surrounding the observed electrode 5, and the electrodes 17, 20, 21, forming a second ring arc 114, illustrated dashed, of immediate neighbour electrodes of and surrounding the first ring arc 112 of electrodes.

According to a first embodiment of the compression preprocessing method, for each observed electrode i, the prediction function fi of the second term of the preprocessed signal is a first invertible prediction function f1ᵢ which depends exclusively on the raw signals observed in the near past up to the instant t_{N-p(i)}, at the integer number m(i) of neighbour electrodes σᵢ(j) of the observed electrode i.

In this case, the expression for the preprocessed signal may be written:

$${ɛ}_{i} ( {t}_{N} ) = {u}_{i} ( {t}_{N} ) - f {1}_{i} ( {( {u}_{{σ}_{i} ( j )} ( {t}_{N - k} ) )}_{{j ∈ [ 1 , m ( i ) ]}_{k ∈ [ 1 , p ( i ) ]}} ) , \qquad Equation 1$$

in which:

t_{N} designates a current sampling instant and N designates the associated current sampling rank;

i is the index of the observed electrode;

uᵢ(t_{N}) represents the raw signal observed at the observed electrode i at the current sampling instant t_{N};

εᵢ(t_{N}) represents the preprocessed signal or error signal at the observed electrode i at the current sampling instant t_{N};

m(i) is the total number of electrodes of a relevant neighbourhood of influence on the observed electrode i, that is to say neighbour electrodes whose raw signals u_{σ}_{ᵢ}₍ⱼ₎(t_{N-k}) influence and contribute to the raw signal of the observed electrode i uᵢ(t_{N}) at the instant t_{N};

σᵢ is a neighbourhood function defining a one-to-one correspondence between an index j of running of the electrodes of the relevant neighbourhood and the identification indices of these electrodes within the total set of the L acquisition electrodes;

j is the running index of the relevant neighbourhood function σᵢ of the observed electrode i, j varying from 1 to m(i);

p(i) designates a depth of memory or of the most distant past rank of the past samples u_{σ}_{ᵢ}₍ⱼ₎(t_{N-k}) of the raw signals of the relevant neighbourhood of the observed electrode i having an influence on the raw signal uᵢ(t_{N}) having the current sampling rank N for the observed electrode i, p(i) depending on the index i if appropriate;

k designates a relative index of running and return to the past of the past samples having an influence on or a contribution to the current sample uᵢ(t_{N}) of the observed raw signal of current rank N.

According to a second embodiment of the compression preprocessing method, for each observed electrode i, the prediction function fi of the second term of the preprocessed signal is a first invertible prediction function f2ᵢ which depends exclusively on the raw signals observed in the near past up to the instant t_{N-p(i)}, at the integer number m(i) of neighbour electrodes σᵢ(j) of the observed electrode i and at the observed electrode i.

In this case, the expression for the preprocessed signal may be written:

$${ɛ}_{i} ( {t}_{N} ) = {u}_{i} ( {t}_{N} ) - f {2}_{i} ( {( {u}_{{σ}_{i} ( j )} ( {t}_{N - k} ) )}_{{j ∈ [ 1 , m ( i ) ]}_{k ∈ [ 1 , p ( i ) ]}} ; {{u}_{i} ( {t}_{N - k} )}_{k ∈ [ 1 , p ( i ) ]} ) , \qquad Equation 2$$

in which:

the influences of the samples of the past of the raw signal of the electrode i, that is to say the contributions of these past samples uᵢ(t_{N-k}), k varying from 1 to p(i) to the raw signal observed at the observed electrode i at the current sampling instant t_{N}, are also taken into account.

According to a third embodiment of the compression preprocessing method, derived from the first embodiment, for each observed electrode i, the prediction function fi of the second term of the preprocessed signal is a first invertible linear prediction function f1Lᵢ which depends exclusively on the raw signals observed in the near past up to the instant t_{N-p(i)}, at the integer number m(i) of neighbour electrodes σᵢ(j) of the observed electrode i.

In this case, the expression for the preprocessed signal may be written:

$${ɛ}_{i} ( {t}_{N} ) = {u}_{i} ( {t}_{N} ) - {\sum }_{k = 1}^{p ( i )} {\sum }_{j = 1}^{m ( i )} {α}_{ijk} * {u}_{{σ}_{i} ( j )} ( {t}_{N - k} ) , \qquad Equation 3$$

in which the aᵢⱼₖ, j varying from 1 to m(i) and k varying from 1 to p(i) are the transformation coefficients of the first linear prediction function f1Lᵢ.

According to a fourth embodiment of the compression preprocessing method, derived from the second embodiment, for each observed electrode i, the prediction function fi of the second term of the preprocessed signal is a second invertible linear prediction function f2Lᵢ which depends on the raw signals observed in the near past up to the instant t_{N-p(i)}, at the integer number m(i) of neighbour electrodes σᵢ(j) of the observed electrode i and at the observed electrode i.

In this case, the expression for the preprocessed signal may be written:

$${ɛ}_{i} ( {t}_{N} ) = {u}_{i} ( {t}_{N} ) - {\sum }_{k = 1}^{p ( i )} ( {a}_{i 0 k} * {u}_{i} ( {t}_{N - k} ) + {\sum }_{j = 1}^{m ( i )} {a}_{ijk} * {u}_{{σ}_{i} ( j )} ( {t}_{N - k} ) ) , \qquad Equation 4$$

in which the aᵢⱼₖ, j varying from 1 to m(i) and k varying from 1 to p(i), and the aᵢ₀ₖ, k varying from 1 to p(i) are the transformation coefficients of the second linear prediction function f2Lᵢ.

As a variant of the four embodiments of the compression preprocessing method described hereinabove, at least one of the integers m(i), p(i) is independent of the index i of the observed electrode.

As a variant, both integers m(i) and p(i) are independent of the index i of the observed electrode i.

According to a fifth embodiment of the compression preprocessing method, derived from the third embodiment, both integers m(i) and p(i) are independent of the index i of the observed electrode i, and for each observed electrode i, the prediction function fi of the second term of the preprocessed signal is a first invertible linear prediction function f1Lᵢ which depends exclusively on the raw signals observed in the near past up to the instant t_{N-p}, at the fixed integer number m of neighbour electrodes σᵢ(j) of the observed electrode i.

In this case, the transformation carried out for the preprocessing of the raw signals for each acquisition channel i of an observed electrode i reduces essentially to determining a linear combination of the surrounding channels so as to remove the spatial and temporal correlation existing between past samples u_{σ}_{ᵢ}₍ⱼ₎(t_{N-k}) of the raw signals of these surrounding channels σᵢ(j) and the raw signal uᵢ(t_{N}) measured by the acquisition channel i on the electrode i observed i corresponding to the current sampling instant t_{N}.

In this case, the expression for the preprocessed signal εᵢ(t_{N}) may be written:

$${ɛ}_{i} ( {t}_{N} ) = {u}_{i} ( {t}_{N} ) - {\sum }_{k = 1}^{p} {\sum }_{j = 1}^{m} {a}_{ijk} * {u}_{{σ}_{i} ( j )} ( {t}_{N - k} ) \qquad Equation 5$$

According to a particular case of the fifth embodiment, surrounding acquisition channels are channels associated with an integer number m of immediate neighbour electrodes σᵢ(j) of the observed electrode i.

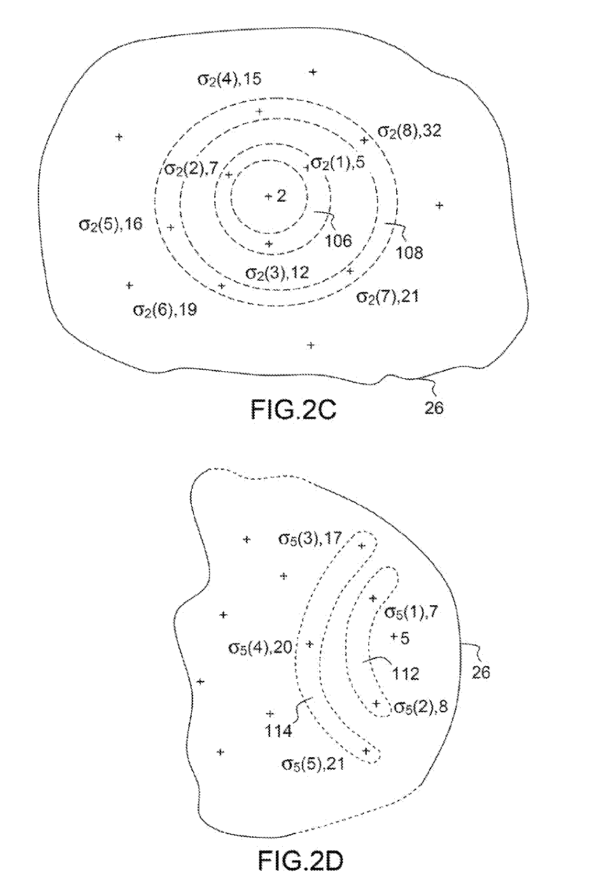

According to FIG. 3, a compression preprocessing method 202 according to the invention comprises a learning step 204 and a step of actual compression preprocessing 206 of the raw data such as is described hereinabove in the diverse embodiments.

The learning step 204 is executed here just once and for all for the set of observed electrodes i, i varying from 1 to L, before the step of transforming the raw data.

In the course of the learning step 204, a set of parameters characterizing the prediction functions fi are determined by adjusting them through a statistical processing, the size of the statistic being dependent on a number of temporal samples which is chosen sufficiently large to minimize the amplitudes of the errors.

When the prediction functions fi are linear transformation functions, it is the coefficients aᵢⱼₖ and/or aᵢ₀ₖ defined in equations 3, 4 and 5 which form the parameters of the linear prediction functions and form linear transformation matrices.

As a variant, the learning step is executed in a manner spread over time per packets of electrodes and in parallel with the step 206 of transforming the raw data into preprocessed data. Stated otherwise, on the basis of an initial suite of parameters characterizing the prediction functions, the parameters and through them the prediction functions are refined.

It should be noted that the principle of a method according to the invention of a compression preprocessing tailored to ECoG data is based on the observation that ECoG data vary slowly in the course of time with respect to the sampling frequency and that the signals originating from the various electrodes in a restricted local neighbourhood are strongly correlated.

The efficiency of the compression is evaluated by calculating the entropy of the signal, before and after compression. The entropy reflects the informative content of the signal: the smaller the entropy, the higher will be the compression rate obtained after application of an entropy encoding.

For example, when we are limited to a sampling on 12 bits, the entropy H of a signal is defined by the expression:

$$H = - {\sum }_{i = 1}^{{2}^{12}} {P}_{s} ln ( {P}_{s} ) , \qquad Equation 6$$

in which

s designates here a symbol taken from among the 2¹² possible vectors of 12 bits of the signal;

Ps designates the probability of occurrence of the symbol s in the time series of the samples of the signal.

According to Shannon's information theorem, the value of the entropy gives the minimum number of bits on which it is necessary to encode the signal so as to retain all its informative content.

The compression preprocessing method which transforms raw data from acquiring electrocorticographic signals on a set of electrodes into a suite of preprocessed data makes it possible to lower the entropy of the raw data, the entropy of the compressed preprocessing data being markedly less than the entropy of the raw data before application of the compression.

It is recalled that in order to retrieve the original data, that is to say the raw data, it suffices to carry out during the preprocessing decompression the operation inverse to the transformation applied during the compression preprocessing since this transformation is invertible.

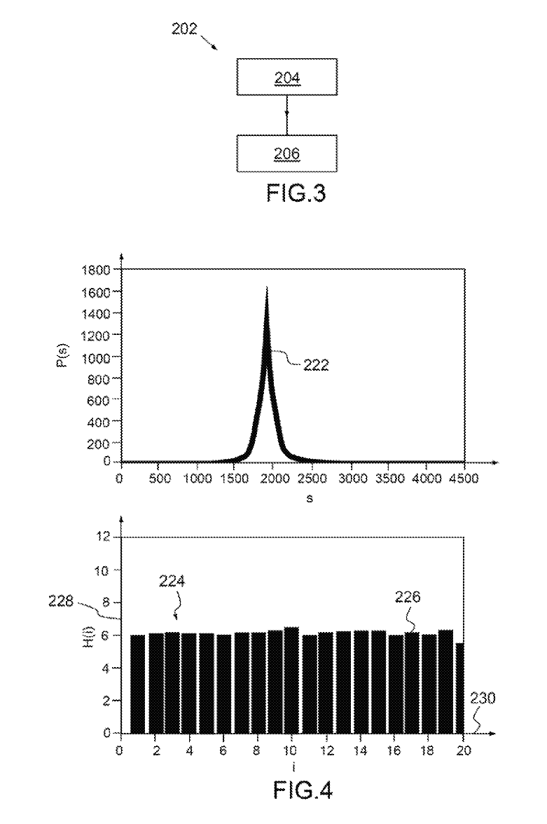

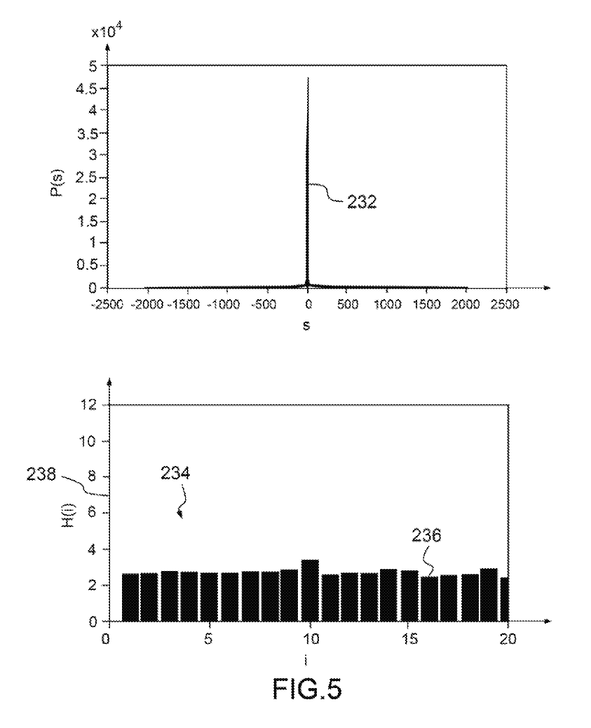

According to FIGS. 4 and 5, a compression algorithm according to the invention has been evaluated on a suite of ECoG data gathered on a monkey by a matrix of cortical electrodes. These data correspond to a recording of a duration of 250 seconds with a frequency of sampling of the observed signals equal to 1 kHz. The data were imported into the Matlab computing tool and then processed.

The processing applied consisted in doing the two steps 204 and 206 of a compression preprocessing method using the transformation function described by equation 5 with p and m fixed equal to 5 and 2 respectively.

In the first step, learning was firstly carried out on uncompressed, that is to say raw, data and the parameters of the prediction functions were extracted.

Next, in the second step, the parameters extracted during the learning step were used to carry out the compression.

According to the top view of FIG. 4, the distribution functions for the symbols of the 20 raw signals acquired by 20 acquisition channels and measured through 20 electrodes appear in the form of a quasi-identical curve 222, thereby showing a uniformity of the laws of distribution of the symbols over the set of acquisition channels.

According to the bottom view of FIG. 4, the evolution of the entropy H(i) of each of the channels is represented in the form of a set 224 of bars 226, this being calculated according to the formula of equation 6 and expressed on the ordinate axis 228, as a function of the numbering of the acquisition channels by a coded integer i on the abscissa axis 230.

It should be noted that the number i of the acquisition channel may be considered to be the identification index of the observed electrode which corresponds thereto.

The entropies H(i) are substantially equal to the value of 6.1 which is equal to the average value of the entropies H(i) over the set of 20 acquisition channels.

According to the top view of FIG. 5, the distribution functions for the symbols of the 20 preprocessed signals corresponding respectively to the raw signals acquired by the 20 acquisition channels appear superimposed in the form of a quasi-identical curve 232, thereby showing a uniformity of the laws of distribution of the symbols of the compressed signals over the set of acquisition channels.

The statistical dispersion of the symbols in these curves is markedly smaller than the statistical dispersion of the symbols which is observed in the curves of the top view of FIG. 4.

According to the bottom view of FIG. 5, the evolution of the entropy H(i) of each compressed signal corresponding to a channel i is represented in the form of a set 234 of bars 236, this being calculated according to the formula of equation 6 and coded on the ordinate axis 238, as a function of the acquisition channel number i represented on the abscissa axis 240.

The entropies H(i) of the preprocessed signals are substantially equal to the value of 2.7 which is equal to the average value of the entropies of the compressed signals over the set of 20 acquisition channels.

Thus the entropy of the set of raw ECoG signals acquired by the set of acquisition channels has been reduced appreciably from 6.1 to 2.7.

The great advantage of a compression preprocessing method using linear prediction functions, in addition to the fact that a high compression rate can be achieved, resides in the fact that the linear operations are not very complex to carry out in terms of the electronic functions required, additions and multiplications, the number of which is governed by the order of the estimation, fixed through the parameters p and m. It is thus possible easily to tailor the complexity of the preprocessing to the calculation power available within the compressor and carry out an optimization of the overall consumption of the transmission unit by altering the ratio R of the compression energy E_{comp}, expended by the compressor, to the energy required for transmission Eₜᵣₐₙₛ divided by the overall compression rate, it being understood that the overall compression rate includes the effect of the preprocessing compression tailored to the ECoG signals and the effect of a generic entropy encoding.

In the case of an absence of compression preprocessing tailored to the raw signals of the application, that is to say corresponding to the case of the entropy-related performance, described in FIG. 4, the maximum compression rate achievable after application of a generic entropy encoding is equal to 49%.

In the case of the use of a compression preprocessing tailored to the raw signals of the application, that is to say corresponding to the case of the entropy-related performance described in FIG. 5, the maximum compression rate achievable after application of a generic entropy encoding is equal to 77%.

It is possible to combine the preprocessing approach according to the invention with other schemes, such as a compression with losses by principal component analysis, wavelet transformations so as to aggregate the advantages of the diverse preprocessing schemes, thus increase the compression rate, but to the detriment of the energy-related performance.

The use of a method of the invention using prediction functions which are not very complex to carry out and which ultimately are not very energy-hungry remains favoured.

All ECoG measurement implants requiring data transmission at high speed or data storage can profit from the type of compression proposed according to the invention since the criterion of low consumption is paramount thereto.

Only the estimation errors εᵢ(t_{N}) of the acquisition channels are transmitted preferably by a radioelectric wireless link or stored efficiently in an electronic memory which is easily implantable in live tissue.

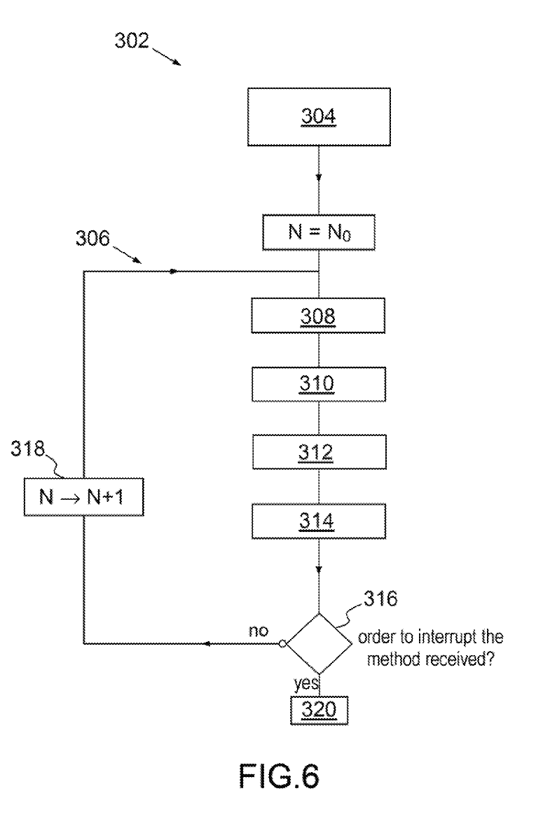

According to FIG. 6, a decompression method 302, corresponding to the compression preprocessing method described hereinabove, comprises a set of steps.

In a first initialization step 304, a suite of initialization data for each observed electrode i is transmitted through a first link and received by the preprocessing decompressor through the transmission receiver.

The initialization data are formed by raw signals, observed in a near past up to the instant t_{N0-p}, p designating the most distant past rank with respect to a rank N₀ of the start instant of the actual implementation of the compression, at an integer number m of neighbour electrodes σᵢ(j) of the observed electrode i, the suite of data being transmitted through a first link.

In an iterative running loop 306 of a current rank N, for the current rank N onwards of N₀, and for each electrode i, the following steps are executed.

In a second step 308 and beforehand, raw signals are provided to the preprocessing decompressor, these raw signals having been transmitted to it directly through the first link or having been reconstructed identically by the preprocessing decompressor itself on the basis of corresponding relevant compressed preprocessing signals, transmitted through a second link.

As a variant and preferably, the second link and the first link form one and the same link.

The raw signals provided are the raw signals observed in a near past up to the instant t_{N-p}, p designating the most distant past rank with respect to the rank N of the current instant, at the integer number m of neighbour electrodes σᵢ(j) of the observed electrode or at the integer number m of neighbour electrodes σᵢ(j) of the observed electrode i and the observed electrode i.

Next, in a third step 310, the compressed preprocessing datum εᵢ(t_{N}) at the sampling instant t_{N} corresponding to the observed electrode i is received by the preprocessing decompressor, it being recalled that the said compressed preprocessing datum has been determined by the implementation of the compression preprocessing method described hereinabove and has been transmitted through the second link.

Thereafter, in a fourth step 312, a second reconstruction term is determined on the basis of the prediction function fi of the second term, applied to the raw signals observed in a near past up to the instant t_{N-p}, at the integer number m of neighbour electrodes σᵢ(j) of the observed electrode i or at the integer number m of neighbour electrodes σᵢ(j) of the observed electrode i and the observed electrode i, the observed raw signals being provided in the providing step 308.

In a fifth step 314, the raw datum of the signal acquired at the sampling instant t_{N} is reconstructed as being equal to the sum of the second reconstruction term and of the preprocessed datum of the signal acquired at the sampling instant t_{N}, at the electrode i.

Next after having verified in a test step 316 that no order to interrupt the decompression method has been received, a unitary incrementation of the current rank N is executed, then the second, third, fourth, fifth steps, 308, 310, 312, 314, and the test step 316 are repeated.

In the case where an order to interrupt the decompression method has been received, the decompression method is interrupted in an interruption step 320.

Thus, the original signal formed by the acquired raw signals can be reconstructed by the preprocessing decompressor, embodied for example by an electronic processor, by carrying out the transformations inverse to the transformations carried out in the course of the preprocessing compression and by adding thereto the estimation errors received forming the transmitted compressed data.

Here, the first link is a wired link and the second link is a radioelectric wireless link.

As a variant and preferably, as represented in FIG. 1, the first and second links form a single radioelectric link for transmitting data.

The system for acquiring and transmitting data of FIG. 1 comprises one or more electronic processors into which a set of instructions forming a computer program are loaded and executed so as to implement the compression method and/or the decompression method which are defined hereinabove.

1. A method of compression preprocessing of raw data uᵢ(t_{N}) of measurements of electrocorticographic signals evolving over time and acquired with the aid of a set of electrodes disposed in direct contact with a cortex and each comprising a different integer identification index i and a position r(i) on the surface of the cortex, the index i varying from 1 to L and L designating the total number of the electrodes,
the raw electrocorticographic temporal signal uᵢ(t) acquired by the electrode of index i being sampled at successive instants t_{N}, N being an integer designating the succession rank of a sample of the signal over time, as a sampled raw signal uᵢ(t_{N}), the coding of whose amplitude forms the raw acquired data of the electrocorticographic raw signal at the sampling instant t_{N},
comprising an actual step of compression preprocessing of the raw signal uᵢ(t_{N}) comprising:
for i varying from 1 to L and at each successive sampling instant t_{N} onwards of a rank N₀, transforming the raw signal acquired uᵢ(t_{N}) by the observed electrode i into a preprocessed signal εᵢ(t_{N}) equal to the difference between a first term and a second term,
the first term being equal to the raw signal uᵢ(t_{N}) acquired at the electrode i at the current instant t_{N}, and
the second term is a prediction function fi which depends temporally on raw signals u_{σ}_{ᵢ}₍ⱼ₎(t_{N-k}), uᵢ(t_{N-k}) observed in a near past up to the instant t_{N-p}, p designating the most distant past rank with respect to the rank N of the current instant, at at least an integer number m of neighbour electrodes σᵢ(j) of the observed electrode i and at most at the integer number m of neighbour electrodes σᵢ(j) of the observed electrode and at the observed electrode i.

2. The method of compression preprocessing according to claim 1, wherein the function fi of the second term is a first prediction function f1ᵢ which depends exclusively on the observed raw signals u_{σ}_{ᵢ}₍ⱼ₎(t_{N-k}) in the near past up to the instant t_{N-p}, at the integer number m of neighbour electrodes σᵢ(j) of the observed electrode i, σᵢ being a neighbourhood function of the observed electrode i.

3. The method of compression preprocessing according to claim 1, wherein the function fi of the second term is a second prediction function f2ᵢ which depends exclusively on the raw signals u_{σ}_{ᵢ}₍ⱼ₎(t_{N-k}), uᵢ(t_{N-k}), observed in the near past up to the instant t_{N-p}, at the integer number m of neighbour electrodes σᵢ(j) of the observed electrode i and at the observed electrode i, σᵢ being a neighbourhood function of the observed electrode i.

4. The method of compression preprocessing according to claim 1, wherein the function fi is a linear prediction function f1Lᵢ which depends exclusively on the raw signals observed in the near past up to the instant t_{N-p}, at the integer number m of neighbour electrodes σᵢ(j) of the observed electrode i or a linear function f2Lᵢ which depends exclusively on the raw signals observed in the near past up to the instant t_{N-p}, at the integer number m of neighbour electrodes σᵢ(j) of the observed electrode and at the observed electrode i.

5. The method of compression preprocessing according to claim 1, wherein
at least one of the integers m, p is independent of the index i of the observed electrode, or
both integers m and p are independent of the index i of the observed electrode.

6. The method of compression preprocessing according to claim 1, wherein the second term is a prediction function which depends on raw signals observed in a near past up to the instant t_{N-p}, p designating the most distant past rank with respect to the rank N of the current instant, at an integer number m of immediate neighbour electrodes σᵢ(j) of the observed electrode i.

7. The method of compression preprocessing according to claim 1, comprising a learning step 206, executed either just once and for all for the set of observed electrodes i, i varying from 1 to L, before the actual step 206 of compression preprocessing of the raw data, or in a manner spread over time per packet of electrodes and in parallel with the actual step 206 of compression preprocessing of the raw data into preprocessed data, and in the course of which a set of parameters comprising the prediction functions fi are determined by adjusting them through a statistical processing, the size of the statistic being dependent on a sufficiently large number of temporal samples.

8. The method of compression preprocessing according to claim 4, wherein the parameters of the linear prediction functions are matrices of coefficients of linear transformations.

9. The method of decompression corresponding to the compression preprocessing method defined according to claim 1, comprising the steps comprising:
in a first initialization step, receiving for each observed electrode i a suite of initialization data formed by raw signals observed in a near past up to the instant t_{N0-p}, p designating the most distant past rank with respect to a rank N₀ of the start instant of the actual implementation of the compression, at an integer number m of neighbour electrodes σᵢ(j) of the observed electrode i, the suite of data being transmitted through a first link; then
in an iterative running loop of a current rank N, for the current rank N onwards of N₀, and for each electrode i,
in a second step, providing raw signals observed in a near past up to the instant t_{N-p}, p designating the most distant past rank with respect to the rank N of the current instant, at the integer number m of neighbour electrodes σᵢ(j) of the observed electrode or at the integer number m of neighbour electrodes σᵢ(j) of the observed electrode i and the observed electrode i, transmitted directly through the first link or reconstructed on the basis of corresponding relevant compressed signals transmitted through a second link; then
in a third step, receiving the preprocessed datum εᵢ(t_{N}) of the signal acquired at the sampling instant t_{N}, at the electrode i, the preprocessed datum having been determined by the implementation of the preprocessing method defined according to claim 1, and having been transmitted through a second link; then
in a fourth step, determining a second reconstruction term on the basis of the prediction function fi of the second term, applied to the raw signals observed in a near past up to the instant t_{N-p}, at the integer number m of neighbour electrodes σᵢ(j) of the observed electrode i or at the integer number m of neighbour electrodes σᵢ(j) of the observed electrode i and the observed electrode i, the observed raw signals being provided beforehand in the second providing step, then
in a fifth step, reconstructing the raw datum uᵢ(t_{N}) of the signal acquired at the sampling instant t_{N}, as being equal to the sum of the second reconstruction term and of the preprocessed datum εᵢ(t_{N}) of the signal acquired at the sampling instant t_{N}, at the electrode i.

10. The method of decompression according to claim 9 wherein,
the first and second links are the same link, or
the first link is a wired link and the second link is a wireless link.

11. A unit for transmitting measurement data of electrocorticographic signals evolving over time comprising a data compression preprocessing unit for converting raw data uᵢ(t_{N}) of measurements of electrocorticographic signals tapped off by electrodes at diverse locations on the surface of a cortex into compressed preprocessing data εᵢ (t_{N}), and a transmission emitter for transmitting the compressed data over a link,
wherein the compression preprocessing unit is configured to implement the compression preprocessing method defined according to claim 1.

12. The unit for transmitting data according to claim 11, wherein the compression preprocessing unit and the transmission emitter are miniaturized so as to be implanted in the body of a patient and the transmission emitter is configured to emit radioelectric waves.

13. A system for acquiring and transmitting data of measurements of electrocorticographic signals evolving over time comprising
a set of electrodes disposed in direct contact with a cortex, each comprising a different integer identification index i and a position r(i) on the surface of the cortex, the index i varying from 1 to L and L designating the total number of electrodes, and each configured to acquire a raw electrocorticographic temporal signal uᵢ(t); and
a sampling unit, configured to sample at successive instants t_{N}, N being an integer designating the succession rank of a sample of a signal over time, the raw signals uᵢ(t) and code their amplitude as associated raw data;
a data compression preprocessing unit for converting the raw data uᵢ(t_{N}) into preprocessed data εᵢ (t_{N}), with lower entropy, and
a transmission emitter for transmitting the compressed preprocessed data;
wherein the compression preprocessing unit is configured to implement an actual step of compression preprocessing of the raw data uᵢ(t_{N}) consisting in: for i varying from 1 to L and at each successive sampling instant t_{N} onwards of a rank N₀, transforming the raw signal uᵢ(t_{N}) acquired by the electrode i into a signal of preprocessed εᵢ (t_{N}) equal to the difference between a first term and a second term, the first term being equal to the signal to the raw signal uᵢ(t_{N}) acquired at the electrode i at the current instant t_{N}, and
the second term being a prediction function fi which depends temporally on raw signals u_{σ}_{ᵢ}₍ⱼ₎(t_{N-k}), uᵢ(t_{N-k}) observed in a near past up to the instant t_{N-p}, p designating the most distant past rank with respect to the rank N of the current instant, at at least an integer number m of neighbour electrodes σᵢ(j) of the observed electrode i and at most at the integer number m of neighbour electrodes σᵢ(j) of the observed electrode and at the observed electrode i.

14. The system for acquiring and transmitting data of measurements of electrocorticographic signals according to claim 13, further comprising:
a transmission receiver, remote from the transmission emitter, for receiving the compressed data.

15. A computer program or product comprising a set of instructions configured to implement the compression preprocessing method defined according to claim 1, when loaded into and executed by a processor.

16. A computer program or product comprising a set of instructions configured to implement the decompression method defined according to claim 9, when loaded into and executed by a processor.

17. A computer program or product comprising a set of instructions configured to implement the compression method and the decompression method according to claim 9, when loaded into and executed by a processor.

18. A system for acquiring and transmitting data of measurements of electrocorticographic signals evolving over time comprising
a set of electrodes disposed in direct contact with a cortex, each comprising a different integer identification index i and a position r(i) on the surface of the cortex, the index i varying from 1 to L and L designating the total number of electrodes, and each configured to acquire a raw electrocorticographic temporal signal uᵢ(t); and
a sampling unit, configured to sample at successive instants t_{N}, N being an integer designating the succession rank of a sample of a signal over time, the raw signals uᵢ(t) and code their amplitude as associated raw data;
a data compression preprocessing unit for converting the raw data uᵢ(t_{N}) into preprocessed data εᵢ (t_{N}), with lower entropy, and
a transmission emitter for transmitting the compressed preprocessed data;
wherein the compression preprocessing unit is configured to implement an actual step of compression preprocessing of the raw data uᵢ(t_{N}) consisting in: for i varying from 1 to L and at each successive sampling instant t_{N} onwards of a rank N₀, transforming the raw signal uᵢ(t_{N}) acquired by the electrode i into a signal of preprocessed εᵢ (t_{N}) equal to the difference between a first term and a second term, the first term being equal to the signal to the raw signal uᵢ(t_{N}) acquired at the electrode i at the current instant t_{N}, and
the second term being a prediction function fi which depends temporally on raw signals u_{σ}_{ᵢ}₍ⱼ₎(t_{N-k}), uᵢ(t_{N-k}) observed in a near past up to the instant t_{N-p}, p designating the most distant past rank with respect to the rank N of the current instant, at at least an integer number m of neighbour electrodes σᵢ(j) of the observed electrode i and at most at the integer number m of neighbour electrodes σᵢ(j) of the observed electrode and at the observed electrode I, further comprising
a transmission receiver, remote from the transmission emitter, for receiving the compressed data, and
a preprocessing decompressor for decompressing the preprocessed data εᵢ (t_{N}) into the raw data uᵢ(t_{N}), configured to execute the steps of the decompression method which are defined according to claim 9.
